(19) United States
(12) Patent Application Publication
(10) Pub. No.: US 2010/0316996 A1
(43) Pub. Date: Dec. 16, 2010

(54) Nasopharyngeal cancer malignancy biomarker and method thereof

(76) Inventors: Lih-Chyang Chen, Xindian City (TW); Yu-Sun Chang, Linkou Township (TW)

Correspondence Address: Muncy, Geissler, Olds & Lowe, PLLC, 4000 Legato Road, Suite 310, FAIRFAX, VA, 22033, US

(21) Appl. No.: 12/456,335

(22) Filed: Jun. 15, 2009

(51) Int. Cl. C12Q 1/68; C07K 14/435

(52) U.S. Cl. 435/6; 530/358

(57) ABSTRACT

The present invention discloses a nasopharyngeal cancer malignancy biomarker and a method thereof, wherein relative hnRNP K expression is used to evaluate the malignancy of nasopharyngeal cancer. The biomarker of the present invention assists the currently-existing inspections to find out cancer in the early stage and realize early diagnosis and early therapy. The present invention also functions as an effective indicator to monitor the metastasis and relapse of nasopharyngeal cancer.

### FIELD OF THE INVENTION

The present invention relates to a technology for testing cancer malignancy, particularly to an hnRNP K-based biomarker for testing the malignancy of nasopharyngeal cancer and a method thereof.

### DESCRIPTION OF THE RELATED ART

Heterogeneous nuclear ribonucleoprotein K (hnRNP K) is a member of the ribonucleoprotein family. HnRNP K can directly interact with DNA and RNA via the K homology domain and can regulate gene expression in different aspects, including transcription, translation and ligation. HnRNP K may induce canceration via regulating the expression of oncogenes c-myc and eIF4E. HnRNP K can respectively join with the internal ribosome entry site of c-myc and the polypyrimidine of the promoter of eIF4E to induce the expression of c-myc and eIF4E. As hnRNP K normally exists in the nucleus, it cannot undertake regulation and translation unless it transfers to cytoplasm. A recent paper pointed out that hnRNP K may cause the metastasis of fibrosarcoma cells. Therefore, hnRNP K is a potential target for metastasis therapy. Some clinical researches pointed out that hnRNP K is abnormally expressed in cells of some specified cancers, including colorectal cancer, esophagus cancer, lung cancer, oral squamous cell cancer, and prostate cancer. The reduced hnRNP K expression in nuclei also correlates with the shorter survival period of the patients of Dukes C colorectal cancer. The association between hnRNP K and other aspects of prognosis, such as the relapse and metastasis of cancer, is not yet defined at present.

Nasopharyngeal cancer prevails in the southeast of China. Nasopharyngeal cancer is sensitive to radiotherapy, but a severe patient of nasopharyngeal cancer still needs chemotherapy in addition to radiotherapy. In current medical technology, one-year survival rate of nasopharyngeal cancer patients is 92%, and five-year survival rate is 50%. About 20-25% patients have distant metastasis. The current inspections for nasopharyngeal cancer include nasopharyngoscopy, endoscopy, cephalic X-ray radiography, and histopathology. However, there is no available nasopharyngeal cancer biomarker to effectively prognose patients, evaluate the overall survival rate of patients, and predict the distant metastasis of cancer cells. There is also no existing molecular medicine addressing over-expressed genes of nasopharyngeal cancer in current medical technology. In the case of hnRNP K, whether it can be used to evaluate the malignancy of nasopharyngeal cancer is not yet defined in the prior-art technology.

### SUMMARY OF THE INVENTION

The primary objective of the present invention is to provide a biomarker for testing the malignancy of nasopharyngeal cancer and a method thereof, wherein hnRNP K (heterogeneous nuclear ribonucleoprotein K) is used to evaluate the malignancy of nasopharyngeal cancer, whereby is selected a correct therapy method and promoted the therapy effect.

To achieve the abovementioned objective, the present invention proposes a nasopharyngeal cancer malignancy biomarker and a method thereof, which is based on the fact that hnRNP K expression is much more abundant in nasopharyngeal cancer tissue than in normal tissue. HnRNP K expression does not correlate with the clinical malignancy indicators, such as the size and depth of cancer tissue, and the stage of lymph metastasis. However, hnRNP K expression is negatively correlated with the overall survival rate, and positively correlated with the distant metastasis of cancer cells. Therefore, the present invention adopts hnRNP K as a biomarker of nasopharyngeal cancer to predict the malignancy of nasopharyngeal cancer and assist the diagnosis and therapy of nasopharyngeal cancer.

Below, the present invention is described in detail in cooperation with the attached drawings to make easily understood the objective, characteristics and accomplishments of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flowchart of a method for testing the malignancy of nasopharyngeal cancer according to the present invention;

FIG. 2 is a flowchart of a method for comparing hnRNP K expression of a testee and a control group according to the present invention;

FIGS. 3A-3C are diagrams showing the distributions of cancer cells in tissue specimens attained according to the present invention; and

FIGS. 4A and 4B are diagrams showing hnRNP K expression in nuclei of the cells of tissue specimens attained according to the present invention.

### DETAILED DESCRIPTION OF THE INVENTION

The present invention addresses the malignancy test of nasopharyngeal cancer. As hnRNP K is highly expressed in nasopharyngeal cancer and clinically correlated with nasopharyngeal cancer, the present invention adopts hnRNP K as a biomarker of nasopharyngeal cancer. Below, a clinical embodiment is used to demonstrate the principle of the present invention and prove the efficacy of the present invention.

Related experiments proved that hnRNP K and the target thereof—thymidine phosphorylase (TP) are over-expressed in nasopharyngeal cancer. The abnormal hnRNP K expression and TP over-expression in cytoplasm correlates with the shorter overall survival period and the distant metastasis. A multivariate analysis shows that hnRNP K and TP in cytoplasm is the independent factors for prognosis.

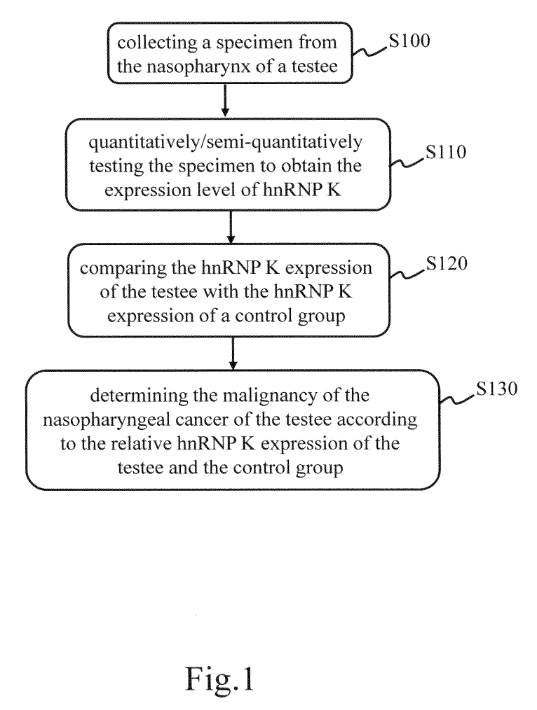

Therefore, the present invention adopts hnRNP K as a biomarker of nasopharyngeal cancer to accurately and effectively test the malignancy of nasopharyngeal cancer. Refer to FIG. 1 a flowchart of a method for testing the malignancy of nasopharyngeal cancer according to the present invention.

Firstly, in Step S100, a specimen is collected from the nasopharynx of a testee. Next, in Step S110, the specimen is quantitatively/semi-quantitatively tested to obtain the expression level of hnRNP K. Next, in Step S120, the hnRNP K expression of the testee is compared with the hnRNP K expression of a control group, which includes negative specimens of nasopharyngeal tissue (specimens of normal nasopharyngeal tissue), positive specimens of nasopharyngeal tissue, and the combinations thereof. Then, in Step S130, the malignancy of the nasopharyngeal cancer of the testee is determined according to the relative hnRNP K expression of the testee and the control group. The specimen in the control group may be a normal nasopharyngeal tissue or a cancerous nasopharyngeal tissue. As the specimens of the control group may be a normal nasopharyngeal tissue or a cancerous nasopharyngeal tissue, the relative expression may present a positive correlation or a negative correlation. The cell lines of nasopharyngeal cancer NPC-TW01, NPC-TW02 and NPC-TW04 were cultivated in DMEM (Dulbecco's Modified Eagle's Medium) supplemented with 10% fetal bovine serum, 100 U/ml penicillin, and 100 μg/ml streptomycin at a temperature of 37° C. in a humidified 5% CO₂ atmosphere. The cells were cultivated in a serum-free DMEM, i.e. treated with a serum deprivation process.

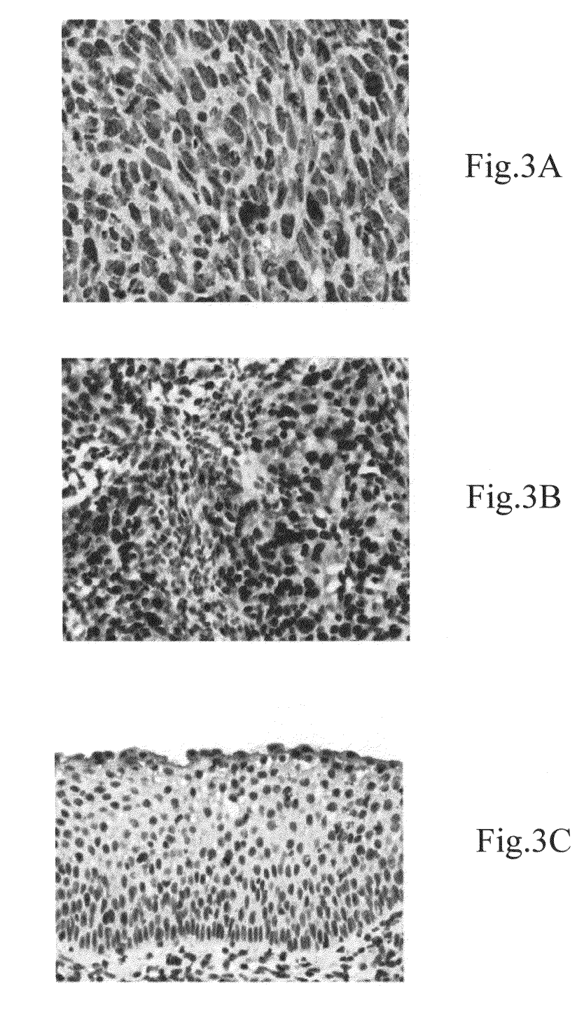

Refer to FIGS. 3A-3C. When hnRNP K is positive in the cytoplasm of over 10% cancer cells of a tissue specimen, hnRNP K is defined to be positive in the cytoplasm of the tissue specimen. When hnRNP K is negative in the cytoplasm of less than or equal to 10% cancer cells of a tissue specimen, the tissue specimen is defined to be negative.

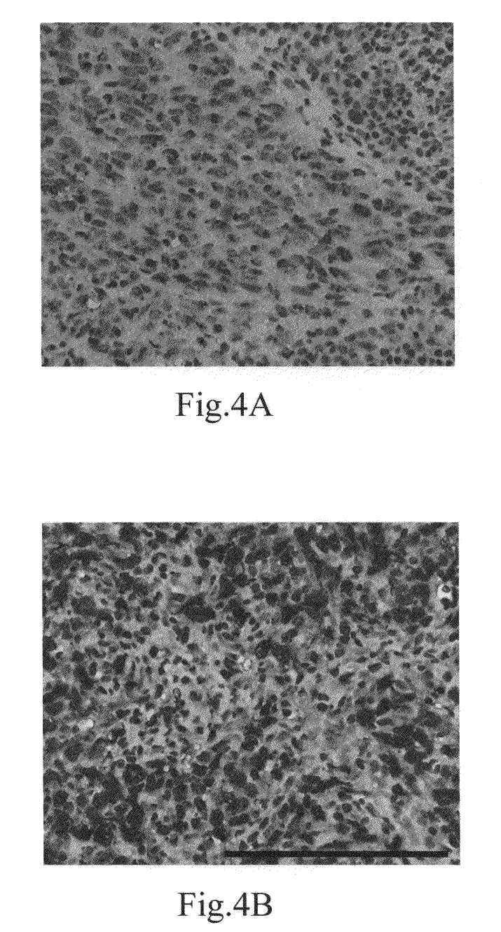

Refer to FIGS. 4A and 4B. When hnRNP K has highly positive expression in the nuclei of over 50% cancer cells of a tissue specimen, the tissue specimen is defined to have high-level hnRNP K expression in the nuclei. When hnRNP K has highly positive expression in the nuclei of less than or equal to 50% cancer cells of a tissue specimen, the tissue specimen is defined to have low-level hnRNP K expression in the nuclei.

Immunohistochemical staining is used in the experiments and performed according to an operation manual of a Bond autostainer (a product of Vision BioSystems, Melbourne, Australia). The retrieval of the tissue slices is undertaken in the Bond Epitope Retrieval Solution 1 (a product of Vision BioSystems). Then, the antibody of hnRNP K is stained. A polymer detection system—the Bond polymer refine (a product of Vision BioSystems) is used to reduce non-specific staining noise. The tissue slices are stained with a liquid DAB reagent, which uses 3′-diaminobenzidine tetrahydrochloride as the chromogen and uses hematoxylin as the counterstaining reagent.

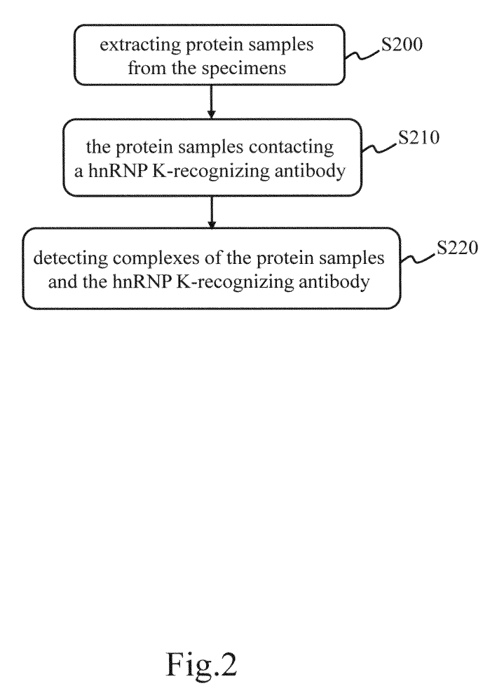

Refer to FIG. 2 a flowchart of a method for comparing hnRNP K expression of the testee and the control group according to the present invention. Firstly, in Step S200, protein samples are extracted from the specimens. Next, in Step S210, the protein samples contact an hnRNP K-recognizing antibody. The hnRNP K-recognizing antibody is a monoclonal antibody or a polyclonal antibody. The protein samples may contact the hnRNP K-recognizing antibody with an immunoassay method. The immunoassay method may be the radioimmunoassay method, the Western blot assay method, the immunofluorescent assay, the enzyme immunoassay, the immunoprecipitation method, the chemiluminescent assay method, the immunohistochemical assay method, the dot blot assay method, or the slot blot assay method. Then, in Step S220, the complexes of the protein samples and the hnRNP K-recognizing antibody are compared.

Herein, the Western blot assay method is used as the exemplification. The mixture solution of the whole cell protein extract and the NP40 lysis buffer solution (50 mM Tris-HCl, pH 7.5, 150 mM NaCl, 1% Igepal CA-630, 1 g/ml Leupeptin, 2 g/ml aprotinin, 1 g/ml pepstatin and 1 mM phenylmethylsulfonyl fluoride) react on ice for 30 minutes. Cell debris is removed from the extract by centrifugation at 12000×g for 10 minutes at a temperature of 4° C., and the supernatant is collected. The concentration of protein is quantitatively measured with the Bradford reagent. Identical amounts (50 g) of protein are separated with electrophoresis and 12% SDS-polyacrylamide gels and then transferred to nitrocellulose membranes. The membranes are blocked with 5% non-fat dry milk in TBS-Tween 20. Nitrocellulose membranes are blocked with 5% non-fat dry milk in TBS-Tween 20, wherein the non-fat dry milk contains the primary anti-TP, anti-hnRNP K, and anti-c-Myc epitope antibodies (Santa Cruz Biotechnology) and the anti-tubulin and anti-actin antibodies (MDBio Inc.). Next, they react at an ambient temperature for 2 hours. Then, the nitrocellulose membranes react with a secondary antibody coupled to horseradish peroxidase and stained with the ECL detection reagents (products of Amersham Pharmacia Biotec).

If the specimens of the control group are negative normal nasopharyngeal tissues, the relative hnRNP K expression is positively correlated with the malignancy of nasopharyngeal cancer. If the specimens of the control group are positive cancerous nasopharyngeal tissues, the relative hnRNP K expression is negatively correlated with the malignancy of nasopharyngeal cancer. If the specimens of the control group are combinations of negative and positive nasopharyngeal tissues, the relative hnRNP K expression has different correlations with the malignancy of nasopharyngeal cancer according to the ratio of negative and positive nasopharyngeal tissues. As abnormal hnRNP K expression in cytoplasm correlates with shorter survival period and distant metastasis, the hnRNP K expression level in the cytoplasm can function as an effective biomarker to diagnose metastasis or predict the metastasis probability of a patient.

The present invention has been demonstrated with the embodiments described above. However, they are only to exemplify the present invention but not to limit the scope of the present invention. Any equivalent modification or variation according to the spirit of the present invention is to be also included within the scope of the present invention, which is based on the claims stated below.

What is claimed is:

1. A method for test malignancy of nasopharyngeal cancer comprising steps:
collecting a specimen from a nasopharynx of a testee;
quantitatively/semi-quantitatively measuring heterogeneous nuclear ribonucleoprotein K (hnRNP K) expression of said specimen;
comparing hnRNP K expression of said specimen of said testee with hnRNP K expression of specimens of a control group; and
predicting malignancy of nasopharyngeal cancer cells of said testee according to relative hnRNP K expression of said testee and said control group.

2. The method for test malignancy of nasopharyngeal cancer of claim 1, wherein said step of comparing hnRNP K expression of said specimen of said testee with hnRNP K expression of specimens of a control group includes
extracting protein samples from said specimens;
said protein samples contacting an hnRNP K-recognizing antibody; and
detecting complexes of said protein samples and said hnRNP K-recognizing antibody.

3. The method for test malignancy of nasopharyngeal cancer of claim 2, wherein said hnRNP K-recognizing antibody is a monoclonal antibody.

4. The method for test malignancy of nasopharyngeal cancer of claim 2, wherein said hnRNP K-recognizing antibody is a polyclonal antibody.

5. The method for test malignancy of nasopharyngeal cancer of claim 2, wherein said protein samples contact said hnRNP K-recognizing antibody with an immunoassay method; said immunoassay method is a method selected from a group consisting of a radioimmunoassay method, a Western blot assay method, a immunofluorescent assay, an enzyme immunoassay, an immunoprecipitation method, a chemiluminescent assay method, an immunohistochemical assay method, a dot blot assay method, and a slot blot assay method.

6. The method for test malignancy of nasopharyngeal cancer of claim 1, wherein said specimens of said control group is selected from the group consisting of negative specimens of nasopharyngeal tissue, positive specimens of nasopharyngeal tissue, and combinations of negative and positive specimens of nasopharyngeal tissue.

7. The method for test malignancy of nasopharyngeal cancer of claim 1, wherein when hnRNP K of said specimen of said testee is over-expressed, said testee is determined to have nasopharyngeal cancer.

8. The method for test malignancy of nasopharyngeal cancer of claim 1, wherein when hnRNP K of said specimen of said testee is over-expressed, said testee is determined to have metastasis of nasopharyngeal cancer or have high probability of metastasis of nasopharyngeal cancer.

9. A nasopharyngeal cancer malignancy biomarker characterized in containing heterogeneous nuclear ribonucleoprotein K (hnRNP K), wherein relative hnRNP K expression of a specimen of a nasopharynx of a testee and specimens of a control group is used to evaluate malignancy of nasopharyngeal cancer of said testee.

10. The nasopharyngeal cancer malignancy biomarker of claim 9, wherein said specimens of said control group is selected from the group consisting of negative specimens of nasopharyngeal tissue, positive specimens of nasopharyngeal tissue, and combinations of negative and positive specimens of nasopharyngeal tissue.

11. The nasopharyngeal cancer malignancy biomarker of claim 9, wherein when hnRNP K of said specimen of said testee is over-expressed, said testee is determined to have nasopharyngeal cancer.

12. The nasopharyngeal cancer malignancy biomarker of claim 9, wherein when hnRNP K of said specimen of said testee is over-expressed, said testee is determined to have metastasis of nasopharyngeal cancer or have high probability of metastasis of nasopharyngeal cancer.
